(19) United States
(12) Patent Application Publication
(10) Pub. No.: US 2025/0090309 A1
(43) Pub. Date: Mar. 20, 2025

(54) BIOFILM MATERIAL, METHOD FOR PREPARING SAME, USE THEREOF, AND ARTIFICIAL HEART VALVE

(71) Applicant: VENUS MEDTECH (HANGZHOU) INC., Hangzhou (CN)

(72) Inventors: Linhe JIN, Hangzhou (CN); Yang HONG, Hangzhou (CN); Dajun KUANG, Hangzhou (CN); Renzheng MA, Hangzhou (CN)

(21) Appl. No.: 18/961,417

(22) Filed: Nov. 26, 2024

Related U.S. Application Data
(63) Continuation of application No. PCT/CN2023/093086, filed on May 9, 2023.

(30) Foreign Application Priority Data
May 27, 2022 (CN) 202210594139.8

(51) Int. Cl. A61F 2/24; A61L 27/36

(52) U.S. Cl. CPC A61F 2/2415; A61F 2/2418; A61L 27/3625; A61L 27/3687; A61L 27/3691; A61L 2430/20

(57) ABSTRACT

Disclosed are a biofilm material, a method for preparing the same, use thereof, and an artificial heart valve. The method for preparating the biofilm material includes: step S100, providing a biofilm; step S200, applying a tensile force in an X direction to the biofilm, wherein the X direction aligns with a fiber direction of the biofilm; and step S300, preforming crosslinking treatment of the biofilm while it is under the tensile force to obtain the biofilm material. In the present disclosure, the tensile force in the same direction as the fiber direction is applied to the biofilm, so that the biofilm is maintained in a stretched and tensioned state, which effectively inhibit the thickening effect of the biofilm during the crosslinking process.

### CROSS-REFERENCE TO RELATED APPLICATIONS

The present application is a Continuation application of PCT Application No. PCT/CN2023/093086, filed on May 9, 2023, which claims the priority of Chinese Patent Application No. 202210594139.8, filed on May 27, 2022, the entire contents of which are hereby incorporated by reference.

### TECHNICAL FIELD

The present disclosure relates to the field of medical device technology, and in particular to a biofilm material, a method for preparing the same, use thereof, and an artificial heart valve.

### BACKGROUND

Heart valve disease causes significant dysfunction of the heart, and when the heart valve is severely impaired, an artificial heart valve is needed to replace the natural valve. Biofilm material is a type of material used to replace impaired or diseased heart valves, mostly derived from animal tissue membranes such as bovine or porcine pericardium. The membrane is subjected to a crosslinking treatment and then connected to a foldable frame structure to form an artificial heart valve, which can be compressed onto a delivery system for implantation into the human body. At present, there are strict requirements for the implanted biofilm material, especially in terms of thickness and mechanical strength. The thickness of the biofilm material directly affects the size of the artificial heart valve during delivery. If the biofilm material is too thick and has poor overall flexibility, it will increase the difficulty of compression, resulting in a larger overall size of the biofilm that is difficult to implant into the target site. Besides, complications may occur during delivery. If the biofilm material is too thin, it has poor tensile properties and is prone to early tearing.

Currently, commercially available biomedical heart valves are generally prepared from crosslinked bovine pericardium, porcine heart valves, and porcine pericardium, which enhance the mechanical properties and prevent post-implantation degradation of the biomaterial. However, some pretreatments to the biofilm material may lead to an increase in its thickness. For example, the crosslinking treatment may significantly thicken the biofilm material, leading to an increase in the size of artificial heart valves, which is not conducive to interventional delivery.

The prior art discloses methods of reducing the thickness of the biofilm material by removing the surface material of the tissue, such as cutting with a dermatome or laser ablation. However, those methods are prone to damaging the tissue structure of the biological valve, resulting in a decrease in mechanical properties. Therefore, there is a need for a method that can implement crosslinking treatment while preventing excessive thickening of biofilm material without damaging the tissue structure of the biological valve.

### SUMMARY

Based on the above problems, the present disclosure provides a method for preparing a biofilm material to effectively adjust the thickness of the biofilm material during the crosslinking process.

The method for preparing the biofilm material of the present disclosure includes:
Step S100, providing a biofilm;
Step S200, applying a tensile force in an X direction to the biofilm, wherein the X direction aligns with a fiber direction of the biofilm; and
Step S300, preforming crosslinking treatment of the biofilm while it is under the tensile force to obtain the biofilm material.

Optionally, the biofilm is derived from pericardium, blood vessels, intestinal mucosa or ligaments. For example, the biofilm is bovine pericardium or porcine pericardium.

Optionally, the biofilm is a sheet structure, a thickness of the biofilm in step S100 is H1, a thickness of the biofilm material in step S300 is H2, and a change rate (H2−H1)/H1 is less than 20%.

Optionally, a thickness of the biofilm in step S100 is H1, a thickness of the biofilm material in step S300 is H2, and the change rate (H2−H1)/H1 is 2% to 15%.

Optionally, the thickness of the biofilm in step S100 is H1, the thickness of the biofilm material in step S300 is H2, and the change rate (H2−H1)/H1 is 5% to 10%.

Optionally, there is at least one area to be treated in the biofilm, the at least one area to be treated has two opposite sides serving as force-bearing portions, and in step S200, the tensile force includes two components that are applied in opposite directions to the two sides.

Optionally, the two sides are located at edge positions of the biofilm.

Optionally, the two sides are parallel to each other.

Optionally, each of the two sides is fixed as a whole or provided with a plurality of fixing sites spaced apart along an extending direction thereof; wherein the plurality of fixing sites on a same side are simultaneously subjected to application of force, or to separately subjected to application of force depending on change in the tensile force.

Optionally, there is at least one area to be treated in the biofilm, the at least one area to be treated has two opposite sides serving as force-bearing portions, and wherein one of the two sides is a stationary edge, other one is an opposite movable edge, and the tensile force is applied to the movable edge; or the two sides are both movable edges, and a tensile force includes two components that are actively applied to each movable edge.

Optionally, wherein each side is clamped or anchored by a connecting member, and the tensile force is applied to the connecting member.

Optionally, the force is applied to the connecting member by a driving mechanism or a counterweight mechanism.

Optionally, the connecting member is a clamp or a thread.

Optionally, in step S200, the tensile force is applied until it reaches a first predetermined value or until a deformation amount of the biofilm reaches a second predetermined value.

Optionally, in step S200, the first predetermined value of the tensile force is 2 to 10N.

Optionally, in step S100, the biofilm has an original first length along the fiber direction, and in step S200, the tensile force is applied to the biofilm until the biofilm has a second length along the fiber direction, with stretching rate of 5% to 15%.

Optionally, in step S300, the biofilm is immersed in a fixative solution for crosslinking treatment, wherein the temperature of the crosslinking treatment is 18 to 26° C., and the period of crosslinking treatment is 6 to 72 hours.

Optionally, the fixative solution is at least one of the following: glutaraldehyde aqueous solution, formaldehyde aqueous solution, ethanol aqueous solution and paraformaldehyde aqueous solution. The formaldehyde aqueous solution is, for example, a neutral formaldehyde aqueous solution.

Optionally, the concentration of the glutaraldehyde aqueous solution is 0.5 wt % to 1.0 wt %.

Optionally, step S200 and step S300 are alternately repeated at least twice, with the tensile force applied increasing sequentially when repeating step S200, until the first predetermined value is reached after a plurality of times of step S200 are performed.

Optionally, step S200 and step S300 are alternately repeated at least twice, with the deformation amount of the biofilm increasing sequentially when repeating step S200, until the second predetermined value is reached after a plurality of times of step S200 are performed.

Optionally, in step S300, the tensile force is maintained by fixing the biofilm to a supporting mechanism.

Optionally, the supporting mechanism is a frame structure including a plurality of side frame strips that enclose a biofilm placement area; wherein at least one of the plurality of side frame strips is movably installed and adjustable in position relative to other side frame strips, or the plurality of side frame strips are fixedly connected; and each side frame strip is installed with a connector that is engageable with the biofilm.

Optionally, an initial length of the biofilm along the fiber direction is D1 and a width of the biofilm is W1. Four side frame strips of the frame structure are fixed to enclose a biofilm placement area with a length of D2 and a width of W2, where D2>D1, W2≥W1. The biofilm is stretched along the fiber direction of the biofilm and then fixed to the frame structure.

The present disclosure further provides a biofilm material prepared by any of the preparation methods.

The present disclosure further provides use of the biofilm material in an artificial heart valve.

The present disclosure further provides an artificial heart valve, including:
a stent of a tubular meshed structure, with an interior of the tubular meshed structure defining a blood flow channel; and
leaflets, mounted within the stent to control an open degree of the blood flow channel, wherein the leaflets are made of the biofilm material according to the disclosure.

The leaflets are fixable to the stent by sewing or other methods, and may further include a membrane covering an inner wall or outer wall of the stent according to functional requirements.

Optionally, edges of each leaflets include a fixed edge fixed to the stent, and a free edge configured to cooperate with free edges of adjacent leaflets in controlling the blood flow channel, and an extending direction of the free edge aligns with the fiber direction of the corresponding leaflet.

The artificial heart valve of the present disclosure can be implanted through catheter intervention or surgery.

Compared with the prior art, the present disclosure has at least the following technical effects.

In the present disclosure, the tensile force is applied to the biofilm in the same direction as the fiber direction thereof to maintain the biofilm in a stretched and tensioned state, and the tensile force can effectively inhibit the thickening effect of the biofilm during the crosslinking treatment.

By maintaining the direction of the tensile force and adjusting the magnitude of the tensile force, it is possible to inhibit the thickening of the biofilm while avoiding the destruction of the collagen fiber structure of the biofilm, thereby ensuring its performance.

### BRIEF DESCRIPTION OF DRAWINGS

FIG. 1 is a flow chart of a preparation method for a biofilm material according to the present disclosure;

FIG. 2 shows a schematic diagram of applying a tensile force to a biofilm in one embodiment;

FIG. 3 shows a schematic diagram of applying a tensile force to a biofilm in another embodiment;

FIG. 4 shows a schematic diagram of applying a tensile force to a biofilm in another embodiment;

FIG. 5 shows a schematic diagram of applying a tensile force to a biofilm in another embodiment;

FIG. 6 shows a schematic diagram of applying a tensile force to a biofilm fixed to a supporting mechanism;

FIG. 7 shows a schematic diagram of the biofilm in FIG. 6 after the tensile force is applied thereto;

FIG. 8 is a schematic diagram showing a framework structure fixing a biofilm in one embodiment;

FIG. 9 is a structural schematic diagram of an artificial heart valve in one embodiment.

The reference signs in the figures are described as follows:
100, biofilm; 110, stationary edge; 120, movable edge; 130, fixing site;
200, connecting member;
300, support mechanism; 310, first side frame strip; 320, second side frame strip; 330, third side frame strip; 340, fourth side frame strip;
400, stent;
500, leaflet; 510, fixed edge; 520, free edge;
X, direction of the tensile force; Y, fiber direction; W, applied width of the tensile force.

### DESCRIPTION OF EMBODIMENTS

The technical solutions according to the embodiments of the present disclosure will be described clearly and fully in combination with the drawings according to the embodiments of the present disclosure. Obviously, the described embodiments are not all embodiments of the present disclosure, but only part of the embodiments of the present disclosure. Based on the disclosed embodiments, all other embodiments obtained by those skilled in the art without creative work fall into the scope of this invention.

It should be noted that, when a component is “connected” with another component, it may be directly connected to another component or may be indirectly connected to another component through a further component. When a component is “provided” on another component, it may be directly provided on another component or may be provided on another component through a further component.

Biofilm material is a type of material used to replace impaired or diseased heart valves, mostly derived from the membranes of animal tissue. The membrane is crosslinked and connected to a foldable frame structure to form an artificial heart valve, which can be compressed onto a delivery system for implantation into the human body. However, crosslinking treatment leads to significant thickening of the biofilm material, reducing the density of collagen fibers per unit volume, and causing the collagen fiber breakage, which results in a decrease in its mechanical properties. If destructive methods such as dermatome cutting and laser ablation are used to reduce the thickness of the biofilm raw material or biofilm material, the mechanical properties of the biofilm material will be even more affected.

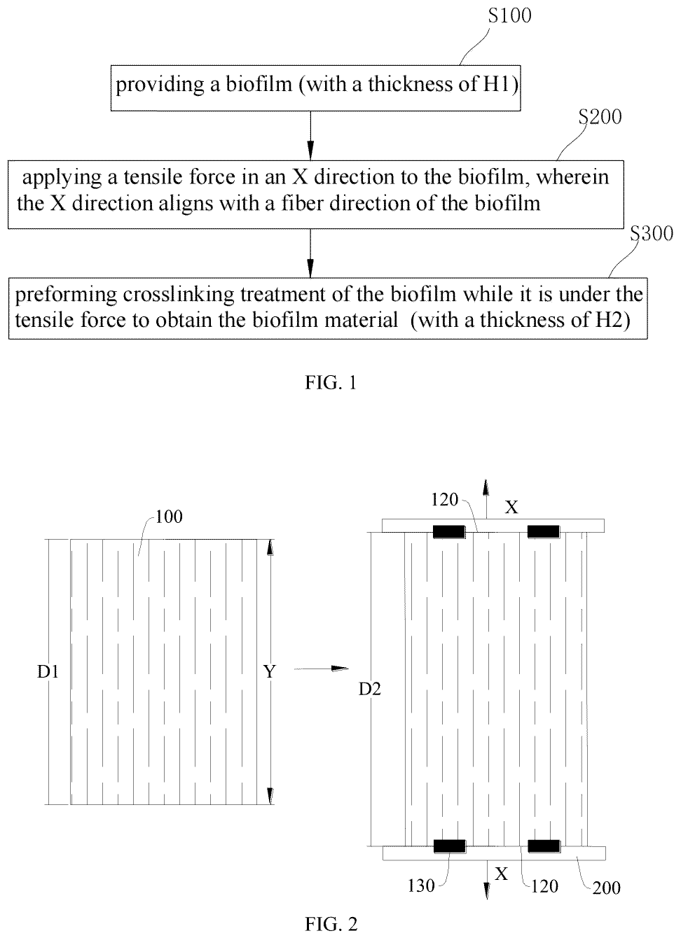

Referring to FIGS. 1 to 7, the present disclosure provides a method for preparing a biofilm material to solve the above problems, including the following steps:

Step S100: providing a biofilm 100;

Step S200: applying a tensile force in the X direction to the biofilm 100, where

the X direction aligns with the fiber direction Y of the biofilm 100; and

Step S300: preforming crosslinking treatment of the biofilm 100 while it is under the tensile force to obtain the biofilm material.

Among them, the biofilm 100 in step S100 is in a first state with an initial thickness H1. To adjust the thickness of the biofilm 100 during the crosslinking process, before the crosslinking treatment (step S300), a tensile force in the X direction is applied to the biofilm 100 (step S200). Under the action of the tensile force, the biofilm 100 is in a stretched and tensioned state, which maintains the flatness of the biofilm 100 and prevents it from curling. This not only improves the uniformity of the crosslinking treatment, but also effectively inhibits the thickening effect caused by the crosslinking treatment in step S300.

Furthermore, by controlling the direction X of the tensile force to align with the fiber direction Y (consistent means that the direction X of the tensile force is roughly parallel to the fiber direction Y, allowing for a slight deviation, such as an angle deviation of less than 10 degrees) in step S200, it is possible to avoid damaging the collagen fiber structure of the biofilm 100 during the crosslinking treatment, ensuring the mechanical properties of the biofilm material. According to practical application requirements, by adjusting the magnitude of the tensile force applied in step S200 and the crosslinking treatment method in step S300, the ideal biofilm material may be obtained, that is, the thickness H2 and mechanical properties of the biofilm material can meet the application requirements.

Among them, the fiber direction Y may be determined by visually observing the shape of the surface of the biofilm 100, or by using a biaxial tensile test method to determine the tensile strength in two perpendicular directions at different angles. The direction with the greatest tensile strength is the direction in which most fibers are oriented, referred as the fiber direction Y. The biaxial tensile test method is a non-destructive experimental method that can determine the fiber direction Y of biofilm 100 before treatment, without affecting the treatment effect and mechanical properties of biofilm 100.

Considering biocompatibility, the biofilm 100 is generally derived from biological tissue membranes, such as membranes of active tissues or organs such as pericardium, blood vessels, intestinal mucosa or ligaments, and the commonly used pericardium is bovine pericardium or porcine pericardium.

The biofilm 100 is a sheet structure, which is conducive to ensuring the uniformity of the treatment as well as ease of application. Compared to the thickness H1 of the biofilm 100 in step S100, the thickness of the biofilm material obtained in step S300 is H2, and the change rate (H2−H1)/H1 is less than 20%. This change rate indicates not only the thickening effect, that is, the thickening rate; when the change rate is a negative value, it also indicates a thinning effect, that is, the thinning rate. If the biofilm is directly crosslinked (with the same crosslinking parameters) without stretching, the prepared biofilm material will be thickened by at least 20%, for example 20% to 25%, compared to the biofilm 100 under the initial state. It can be seen that compared to direct crosslinking without stretching of the biofilm, the preparation method of the present disclosure can effectively inhibit the thickening effect of the biofilm 100 during the crosslinking process, and even bring about a thinning effect.

The biofilm 100 may be pre-treated, for example, by removing cells or excess tissues, before crosslinking treatment. For example, in step S100, the provided biofilm 100 is pre-cut to have a more regular shape, so as to facilitate the application of tensile force to the biofilm 100.

In step S300, the crosslinking treatment is performed by immersing the biofilm 100 in a fixative solution. The fixative solution may be at least one of the following: glutaraldehyde aqueous solution, formaldehyde aqueous solution, ethanol aqueous solution, and paraformaldehyde aqueous solution, wherein the formaldehyde aqueous solution may be a neutral formaldehyde aqueous solution. The crosslinking treatment temperature is 18 to 26° C., and the period of crosslinking treatment is 6 to 72 hours.

In step S300, a glutaraldehyde aqueous solution with a concentration of 0.5 wt % to 1.0 wt % may be used. For example, a glutaraldehyde aqueous solution with a concentration of 0.625% can reduce the residual glutaraldehyde while ensuring the fixation effect.

Changes of the biofilm material in step S300 compared to that of the biofilm 100 in step S100 include but are not limited to the thickness and mechanical properties. In terms of thickness, the biofilm 100 in step S300 has a thickening rate of 2 to 15% compared to the biofilm in step S100, and the thickening rate within this range will not significantly increase the shrinkage size of the biofilm material. For example, in one embodiment, the thickening rate is 5% to 10%, and for another example, the thickening rate is 6.45%.

In order to effectively apply the tensile force to the biofilm 100, there is at least one area to be treated in the biofilm 100, the at least one area to be treated has two opposite sides acting as force-bearing portions. In step S200, the tensile force includes two opposite components that may be applied to the two sides to put the biofilm 100 in a stretched and tensioned state, wherein the two opposite sides refer to sides of the area to be treated along the fiber direction Y, so that the direction of each component of the tensile force applied to the two sides aligns with the fiber direction Y of the biofilm 100, avoiding damage to the fiber structure of the biofilm 100.

Furthermore, the two sides are parallel to each other, which facilitates maintaining the direction of the tensile force, as shown in FIGS. 2-5.

In one embodiment, the two side edges are at the edge positions of the biofilm 100, so as to maximize the area to be treated of the biofilm 100 and improve its utilization rate.

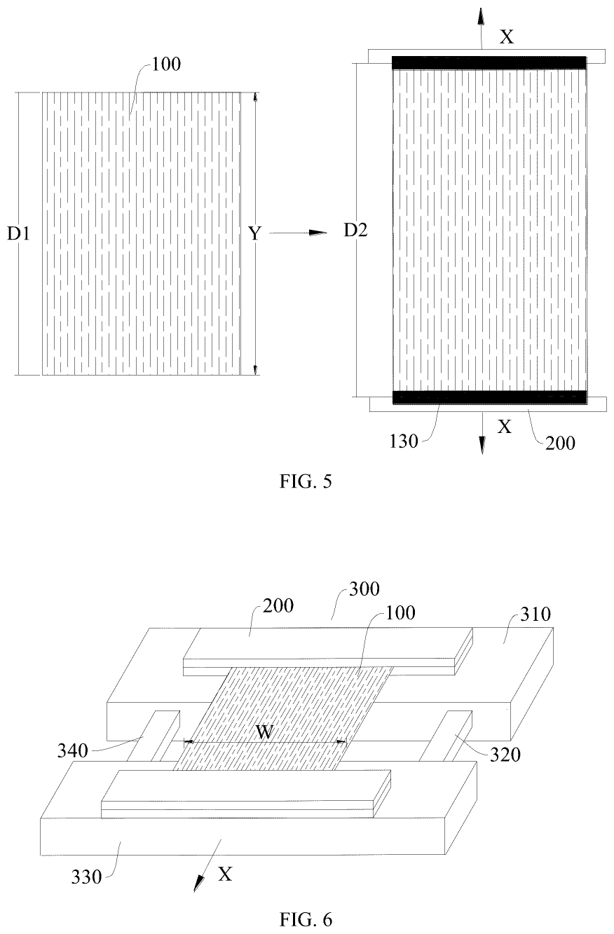

Before applying the tensile force, it is necessary to set force point on each side, which can be set up in various ways. For example, each side may be fixed as a whole, as shown in FIG. 5, and the operation is relatively simple. Multiple fixing sites 130 (FIGS. 2-4), understood as portions of the biofilm directly subjected to force, may also be arranged at intervals along the extending direction of the side. In case the plurality of fixing sites are continuously distributed rather than arranged at intervals, it may also be interpreted as fixing as a whole.

The plurality of fixing sites 130 can provide more force points, making it easier to adjust the force applied to each fixing site 130 and avoiding local tearing of the biofilm 100 due to excessive force, which is more suitable for circular biofilms 100 or biofilms with less regular shapes. The above two methods each have their advantages and may be selected according to the actual shape of the biofilm 100 and application requirements.

Referring to FIG. 4, one of the two sides is a stationary edge 110 and the other is an opposite movable edge 120. The tensile force is applied to the movable edge 120 to cause the biofilm 100 to deform along the extending direction of the movable edge 120, where the deformation amount may be quantified in terms of the stretching rate, which is directly affected by the magnitude of the tensile force.

In another embodiment, both sides are movable edges 120, and the tensile force includes two components that are actively applied to each of the movable edges 120 (see FIG. 2), so that the biofilm 100 is stretched towards both sides and deformed, which is beneficial for the overall force uniformity of the biofilm.

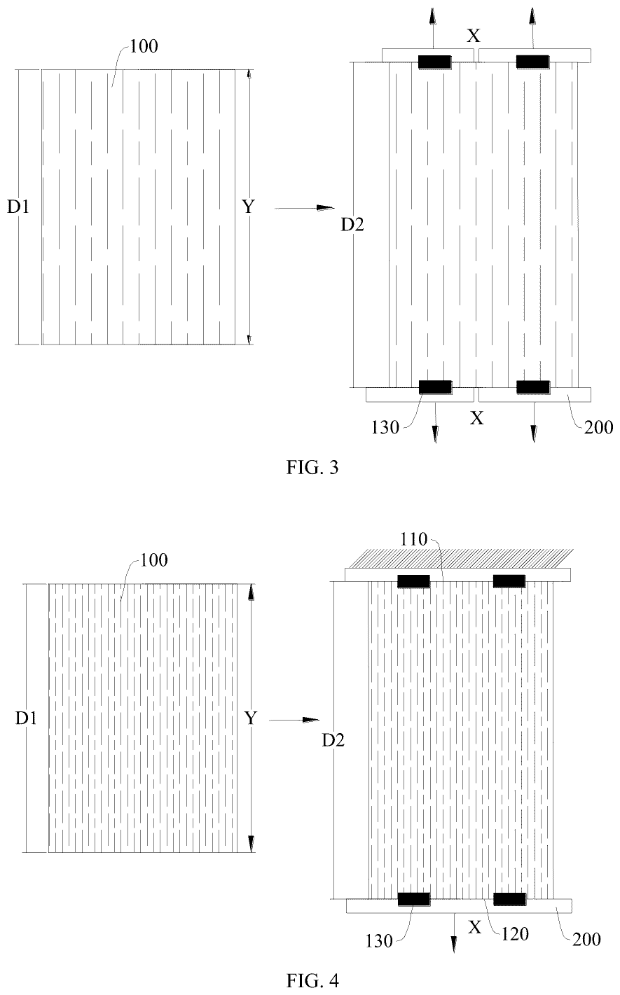

In the case where there are multiple fixing sites 130 on each side, there are various ways to apply the tensile force, for example, the force is applied simultaneously to multiple fixing sites 130 on the same side (see FIG. 2) to ensure the stretching uniformity of the side. Considering the unevenness of the biofilm 100, the force may also be respectively applied to the fixing site according to the change of tensile force (see FIG. 3), to avoid excessive stretching and tearing of each side.

In step S200, when applying the tensile force, the connecting member 200 may be used to clamp or anchor the side of the biofilm. Whether the side is clamped (the connecting member does not penetrate the biofilm) or anchored (the connecting member penetrates the biofilm), it can be fixed as a whole or by multiple fixing sites 130, as shown in FIGS. 2 to 7. The connecting member 200 may be a clamp or a thread, etc. By applying force to the connecting member 200, the tensile force can be applied to the biofilm 100.

The method of applying force to the connecting member 200 mentioned above may be achieved by a driving mechanism or a counterweight mechanism, whereby the biofilm 100 is hung vertically and stretched under the action of gravity, the source of which may be achieved by adding weights and other means.

In step S200, it is required to apply the tensile force to the biofilm until it reaches a first predetermined value, or the deformation amount of the biofilm 100 reaches a second predetermined value, so as to effectively inhibit the thickening effect of the biofilm 100 caused by the crosslinking treatment in step S300.

The first predetermined value of the tensile force is related to the applied width W (perpendicular to the fiber direction Y) of the biofilm 100. For example, when the applied width is 5 to 10 cm, the first predetermined value of the tensile force applied is 0 to 10 N (excluding 0), wherein the dimensional changes are converted proportionally, so that the biofilm is kept in a stretched and tensioned state without damaging the fiber structure of the biofilm 100. Specifically, referring to FIG. 6, the applied width W of the tensile force is 8 cm, and the first predetermined value of the tensile force is 2 to 10 N.

The deformation amount of the biofilm 100 may be characterized in terms of the stretching rate of the biofilm 100 along the fiber direction Y. For example, in step S100, the biofilm 100 is in the first state with a first length D1 along the fiber direction Y, and after the biofilm 100 is stretched along the fiber direction Y in step S200, the edge of the biofilm along the fiber direction Y has a second length D2. In order to effectively control the thickness of the biofilm material, it is generally required that the deformation amount (stretching rate) of the biofilm 100 be in the range of 0 to 15%, such as 5 to 15%, to ensure that the thickening rate of the biofilm 100 can be controlled within 20%.

In the preparation method of the present disclosure, another processing method is to alternately repeat step S200 and step S300 at least twice, with the tensile force applied increasing sequentially when repeating step S200, until the tensile force reaches the first predetermined value, or the deformation amount of the biofilm 100 reaches a second predetermined value, and the alternation is completed. If the first predetermined value of tensile force is applied to the biofilm 100 at once, the instantaneous tensile force is too much for the biofilm 100, which has a poorly stabilized fiber structure in the fresh state. This may lead to excessive deformation of the collagen fibers, resulting in hardening of the biofilm and affecting its fluid dynamics performance. However, the alternating mode of the present disclosure can enhance the adaptive ability of the biofilm 100, reduce damage to the biofilm 100, and maintain good mechanical properties of biofilm 100. For example, in one embodiment, the first predetermined value of the tensile force is T, and the force applied in the initial step S200 is 0.2T to 0.5T. During the alternating process, the tensile force applied increases by 0.1T to 0.5T sequentially when repeating step S200.

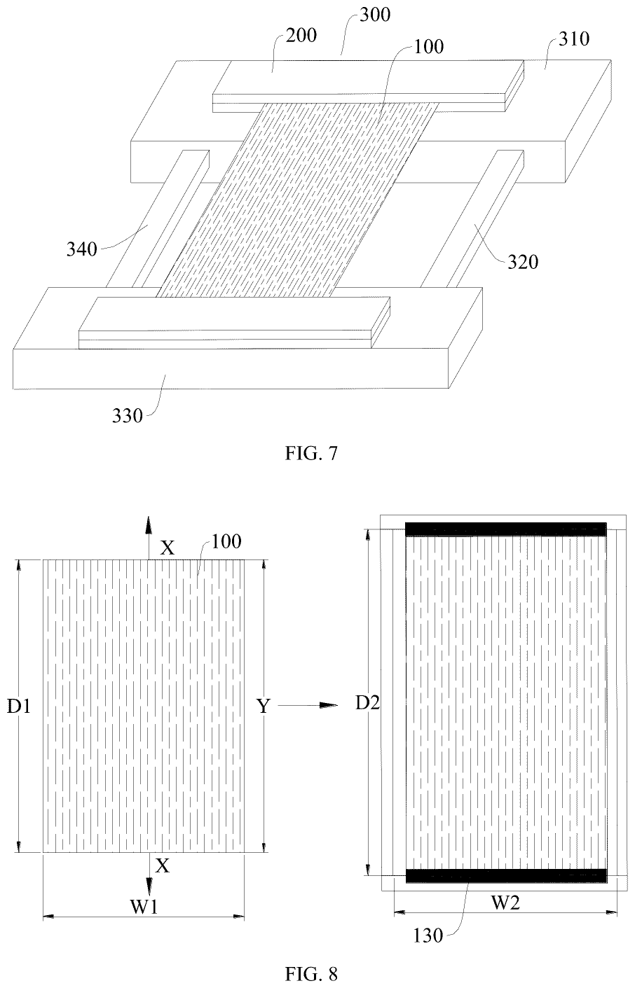

In step S300, the tensile force is maintained by fixing the biofilm 100 after applying the tensile force to the support mechanism 300, so as to alternately repeat step S200 and step S300. The support structure 300 may be a frame structure, which typically includes multiple side frame strips that enclose a biofilm placement area. At least one side frame strip is movable and adjustable relative to the other side frame strips. Referring to FIG. 6, the support mechanism 300 has four side frame strip strips, namely the first side frame strip 310, the second side frame strip 320, the third side frame strip 330 and the fourth side frame strip 340. The first side frame strip 310 is fixed arranged, while the second side frame strip 320 and the fourth side frame strip 340 are movably arranged, that is, adjustable in position relative to the first side frame 310. The biofilm 100 is fixed between the first side frame strip 310 and the third side frame strip 330 by the connecting member 200, therefore, the tensile force applied in the X direction to the third side frame strip 330 can stretch the biofilm 100 along the fiber direction, see FIG. 7.

Referring to FIG. 8, the initial length of the biofilm 100 along the fiber direction is D1, and the width is W1. The four side frame strips of the frame structure are fixed relative to each other, enclosing the biofilm placement area with a length of D2 and a width of W2, where D2>D1, W2≥W1. During operation, the biofilm 100 is stretched along the fiber direction, for example, manually or with the aid of other tooling. After being stretched to a sufficient length, the two ends of the biofilm 100 along the fiber direction are fixed to the frame structure, and the two ends along the width direction may be fixed or free. According to the expected deformation amount (preset stretching rate), frame structures of different sizes may be selected.

There are many ways to fix the biofilm 100 to the frame structure. In one embodiment, the connecting member 200, which engages with the biofilm 100, is arranged on the frame structure, for example, installed on each side frame strip to facilitate the fixation and detachment of the biofilm 100. The connecting member may be a clamp or a thread.

The present disclosure further provides a biofilm material prepared by any of the above preparation methods.

In the prior art, the biofilm material prepared by crosslinking without stretching is generally thickened by at least 20% compared to the biofilm 100 under the initial state. However, the thickness of the biofilm material prepared according to the preparation method of the present disclosure increases by no more than 20% compared to that of the biofilm under the initial state (step S100). For example, in one embodiment, the thickness of the biofilm material increases by 6.45% compared to that of the biofilm under the initial state (step S100).

The present disclosure further provides the use of the above-mentioned biofilm material in artificial heart valves, which may be implanted into the body through catheter intervention or surgical operation. Compared with the biofilm material obtained by direct crosslinking and fixation, the preparation method of the present disclosure can effectively inhibit the thickening effect of the biofilm 100 during the crosslinking treatment, and even bring about thinning effect of the biofilm, which is conducive to reducing the overall size of the artificial heart valve, making it adapted to a smaller caliber delivery system and minimizing the damage to the blood vessels during the transcatheter interventional surgery.

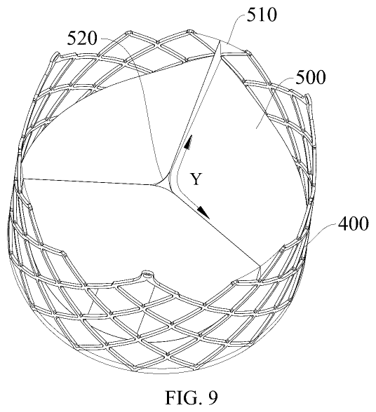

Referring to FIG. 9, the present disclosure further provides an artificial heart valve, including a stent 400 and leaflets 500, wherein the stent 400 is a tubular meshed structure, and the interior thereof defines a blood flow channel. The leaflets 500 are installed within the stent 400 to control the open degree of the blood flow channel. The leaflets 500 are prepared by the preparation method described above and can meet the requirements of free opening and closing in accordance with the blood flow, as well as the fatigue performance. Due to the thinner thickness of the leaflet 500 prepared by the above method, it has better fluid dynamics performance. The leaflet 500 may be fixed to the stent 400 by sewing or the like, and according to functional requirements, it may further include a membrane covering the inner wall or outer wall of the stent 400.

The artificial heart valve has at least two leaflets 500. When working, the opening and closing of the blood flow channel is controlled primarily by the edges of the leaflets 500. In one embodiment, edges of each leaflets include a fixed edge 510 fixed to the stent 400 and a free edge 520 disposed between the adjacent leaflets 500, as shown in FIG. 9, wherein the free edge 520 is configured to cooperate with free edges of adjacent leaflets in controlling the blood flow channel, thereby adjusting the open degree of the blood flow channel. In this embodiment, the extending direction of the free edge 520 aligns with the fiber direction Y of the corresponding leaflet 500, so that the artificial heart valve has good mechanical properties, can withstand environmental stresses, and is conducive to prolonged service life.

### Embodiments 1-4

Step S100, a biofilm derived from the porcine pericardium was pre-cleaned and cut into a rectangle shape with a length of D1 along the fiber direction.

Step S200, as shown in FIG. 2, the two ends of the cut pericardium along the fiber direction were fixed to the movable edges of the bidirectional stretcher, respectively, and the pericardium was stretched to a length of D2 along the fiber direction according to the preset stretching rate (wherein the stretching rates in Embodiments 1 to 4 are different, as shown in Table 1), and the bidirectional stretcher was fixed.

Step S300, the pericardium stretched to the length of D2 in step S200 was immersed in a glutaraldehyde solution with a concentration of 0.625 wt % at 20° C. for 48 hours.

### Control Example 1

Step S100, a biofilm derived from the porcine pericardium was pre-cleaned and cut into a rectangle shape with a length of D1 along the fiber direction.

Step S200, the pericardium cut in step S100 was immersed in a glutaraldehyde solution with a concentration of 0.625 wt % at 20° C. for 48 hours.

The elongation rates and the corresponding change rates of pericardium in Embodiments 1 to 4 and the Control Example 1 are shown in Table 1 below.

TABLE 1
The elongation ratios and the corresponding change ratios of
pericardium in Embodiments 1 to 4 and Control Example 1

| | D1 | D2 | Elongation | Change Rate |
|---|---|---|---|---|
| Number | (cm) | (cm) | (%) | (%) |
| Control Example 1 | 20 | — | 0 | 20.18 |
| Embodiment 1 | 19 | 20 | 5.26 | 15.33 |
| Embodiment 2 | 18 | 20 | 11.11 | 6.45 |
| Embodiment 3 | 17.6 | 20.2 | 15 | 2.35 |
| Embodiment 4 | 17 | 20 | 17.65 | −1.06 |

### Embodiments 5-8

Step S100, a biofilm derived from the porcine pericardium was pre-cleaned and cut into a rectangle shape with a length of D1 along the fiber direction.

Step S200, as shown in FIG. 4, the two ends of the cut pericardium along the fiber direction were respectively fixed to the fixed side and the movable side of the unidirectional stretcher, and the pericardium was stretched to a length of D2 along the fiber direction according to the preset stretching rate (wherein the stretching rates in Embodiments 5 to 8 are different, as shown in Table 2), and the unidirectional stretcher was fixed.

Step S300, the pericardium stretched to a length of D2 in step S200 was immersed in a glutaraldehyde solution with a concentration of 0.625 wt % at 20° C. for 48 hours.

### Control Example 2

Step S100, a biofilm derived from the porcine pericardium was pre-cleaned and cut into a rectangle shape with a length of D1 along the fiber direction.

Step S200, the pericardium cut in step S100 was immersed in a glutaraldehyde solution with a concentration of 0.625 wt % at 20° C. for 48 hours.

The elongation rates and the corresponding change rates of pericardium in Embodiments 5 to 8 and Control Example 2 are shown in Table 2 below.

TABLE 2
Elongation and corresponding change rate of pericardium
in Embodiments 5 to 8 and Control Example 2

| | D1 | D2 | Elongation | Change Rate |
|---|---|---|---|---|
| Number | (cm) | (cm) | (%) | (%) |
| Control Example 2 | 18 | — | 0 | 20.18 |
| Embodiment 5 | 18 | 19 | 5.56 | 14.88 |
| Embodiment 6 | 18 | 20 | 11.11 | 6.45 |
| Embodiment 7 | 18 | 20.5 | 13.80 | 2.35 |
| Embodiment 8 | 17 | 20 | 17.65 | −2.37 |

### Embodiments 9-12

Step S100, a biofilm derived from the porcine pericardium was pre-cleaned and cut into a rectangle shape with a length of D1 along the fiber direction and a width of W1.

Step S200, as shown in FIG. 8, the cut pericardium was manually stretched along the fiber direction according to a preset stretching rate (wherein the stretching rates in Embodiments 9 to 12 are different, as shown in Table 3), and fixed to a rectangular biofilm placement area with a length of D2 and a width of W2.

Step S300, the pericardium stretched to a length of D2 in step S200 was immersed in a glutaraldehyde solution with a concentration of 0.625 wt % at 20° C. for 48 hours.

### Control Example 3

Step S100, a biofilm derived from the porcine pericardium was pre-cleaned and cut into a rectangle shape with a length of D1 along the fiber direction and a width of W1, and fixed to a rectangular biofilm placement area with a length of D1 and a width of W1.

Step S200, the pericardium cut in step S100 was immersed in a glutaraldehyde solution with a concentration of 0.625 wt % at 20° C. for 48 hours.

The elongation rates and the corresponding change rates of pericardium in Embodiments 9 to 12 and Control Example 3 are shown in Table 3 below.

TABLE 3
Elongation and corresponding change rate of pericardium
in Embodiments 9 to 12 and Control Example 3

| | D1 | D2 | Elongation | Change Rate |
|---|---|---|---|---|
| Number | (cm) | (cm) | (%) | (%) |
| Control Example 3 | 18 | — | 0 | 20.18 |
| Embodiment 9 | 19 | 20 | 5.26 | 14.68 |
| Embodiment 10 | 18 | 20 | 11.11 | 6.14 |
| Embodiment 11 | 17.5 | 20 | 14.29 | 2.03 |
| Embodiment 12 | 17 | 20 | 17.65 | −2.68 |

The aforementioned embodiments only represent several embodiments of the present disclosure, and the descriptions thereof are relatively specific and detailed, but should not be construed as a limitation on the scope of the patent of the present disclosure. It should be noted that, for a person of ordinary skill in the art, several variations and improvements may be made without departing from the concept of the present disclosure, and these are all within the protection scope of the present disclosure. Therefore, the protection scope of the present disclosure shall be subject to the appended claims.

What is claimed is:

1. A preparation method for a biofilm material, comprising:
Step S100, providing a biofilm;
Step S200, applying a tensile force in an X direction to the biofilm, wherein the X direction aligns with a fiber direction of the biofilm; and
Step S300, preforming crosslinking treatment of the biofilm while it is under the tensile force to obtain the biofilm material.

2. The preparation method of claim 1, wherein the biofilm is a sheet structure, a thickness of the biofilm in step S100 is H1, a thickness of the biofilm material in step S300 is H2, and a change rate (H2−H1)/H1 is less than 20%.

3. The preparation method of claim 2, wherein the change rate (H2−H1)/H1 is 2% to 15%.

4. The preparation method of claim 1, wherein there is at least one area to be treated in the biofilm, the at least one area to be treated has two opposite sides serving as force-bearing portions, and in step S200, the tensile force comprises two components that are applied in opposite directions to the two sides.

5. The preparation method of claim 4, wherein the two sides are located at edge positions of the biofilm and parallel to each other.

6. The preparation method of claim 4, wherein each of the two sides is fixed as a whole or provided with a plurality of fixing sites spaced apart along an extending direction thereof; and
wherein the plurality of fixing sites on a same side are simultaneously applied with force, or separately applied with force based on change in the tensile force.

7. The preparation method of claim 1, wherein there is at least one area to be treated in the biofilm, the at least one area to be treated has two opposite sides serving as force-bearing portions, and
wherein one of the two sides is a stationary edge, other one of the two sides is an opposite movable edge, and the tensile force is applied to the movable edge; or
the two sides are both movable edges, and the tensile force comprises two components that are actively applied to each of the movable edges.

8. The preparation method of claim 5, wherein each side is clamped or anchored by a connecting member, and the tensile force is applied to the connecting member.

9. The preparation method of claim 8, wherein the force is applied to the connecting member by a driving mechanism or a counterweight mechanism.

10. The preparation method of claim 8, wherein the connecting member is a clamp or a thread.

11. The preparation method of claim 1, wherein the step of applying the tensile force comprises applying the tensile force until it reaches a first predetermined value or until a deformation amount of the biofilm reaches a second predetermined value.

12. The preparation method of claim 11, wherein in step S100, the biofilm has an original first length along the fiber direction, and in step S200, the tensile force is applied to the biofilm until the biofilm reaches a second length along the fiber direction, with a stretching rate of 5% to 15%.

13. The preparation method of claim 1, wherein in step S300, the biofilm is immersed in a fixative solution for crosslinking, the fixative solution is at least one of the following: glutaraldehyde aqueous solution, formaldehyde aqueous solution, ethanol aqueous solution, and paraformaldehyde aqueous solution; and wherein a temperature of the crosslinking treatment is 18 to 26° C., and a period of the crosslinking treatment is 6 to 72 hours.

14. The preparation method of claim 11, wherein step S200 and step S300 are alternately repeated at least twice, with the tensile force applied increasing sequentially when repeating step S200, until the first predetermined value is reached after S200 is performed for a plurality of times.

15. The preparation method of claim 11, wherein step S200 and step S300 are alternately repeated at least twice, with the deformation amount of the biofilm increasing sequentially when repeating step S200, until the second predetermined value is reached after S200 is performed for a plurality of times.

16. The preparation method of claim 11, wherein in step S300, the tensile force is maintained by fixing the biofilm to a supporting mechanism.

17. The preparation method of claim 16, wherein the support mechanism is a frame structure comprising a plurality of side frame strips that enclose a biofilm placement area, wherein at least one of the plurality of side frame strips is movably installed and adjustable in position relative to other side frame strips, or the plurality of side frame strips are fixedly connected, and each side frame strip is installed with the connecting member that is engageable with the biofilm.

18. A biofilm material, prepared by the preparation method of claim 1.

19. An artificial heart valve, comprising:
a stent of a tubular meshed structure, with an interior of the tubular meshed structure defining a blood flow channel; and
leaflets, mounted within the stent to control an open degree of the blood flow channel, wherein the leaflets are made of the biofilm material of claim 18.

20. The artificial heart valve of claim 19, wherein edges of each leaflets comprise: a fixed edge fixed to the stent, and a free edge configured to cooperate with free edges of adjacent leaflets in controlling the blood flow channel, and wherein an extending direction of each free edge aligns with a fiber direction of corresponding leaflet.
